(19) United States
(12) Patent Application Publication
(10) Pub. No.: US 2017/0354433 A1
(43) Pub. Date: Dec. 14, 2017

(54) DEVICE FOR PLANTAR FASCIA ENDOSCOPY AND SOFT TISSUE RELEASES

(71) Applicant: Next Step Consulting, LLC, Frisco, TX (US)

(72) Inventor: Dominique Nickson, Frisco, TX (US)

(73) Assignee: Next Step Consulting, LLC, Frisco, TX (US)

(21) Appl. No.: 15/622,067

(22) Filed: Jun. 13, 2017

Related U.S. Application Data
(60) Provisional application No. 62/349,963, filed on Jun. 14, 2016.

(51) Int. Cl. A61B 17/32; A61B 17/34; A61B 17/04; A61B 17/00

(52) U.S. Cl. CPC A61B 17/320016; A61B 17/34; A61B 17/320092; A61B 2017/0409; A61B 2017/0042

(57) ABSTRACT

A clamping arthroscopic resection tool is provided. The clamping arthroscopic resection tool includes a first jaw and a second jaw positionable on opposing sides of a tissue, a clamping structure which biases the first jaw and the second jaw against the tissue to clamp the tissue between the first jaw and the second jaw, an indicator mark disposed along one of the first jaw and the second jaw at an intermediate location, and a cutting member which is movable between the first jaw and the second jaw and between a non-cutting position and a cutting position to cut the tissue up to the indicator mark.

### RELATED APPLICATIONS

The present application claims the benefit of U.S. Provisional Application Ser. No. 62/349,963, filed Jun. 14, 2016, which is herein incorporated by reference in its entirety.

### THE FIELD OF THE INVENTION

The present invention relates to soft tissue releases. In particular, examples of the present invention relate to a device and related method for plantar fascia endoscopy and soft tissue releases.

### BACKGROUND

Some conditions, such as plantar fasciitis, may require a soft tissue release for treatment. Plantar fasciitis involves pain and inflammation of a thick band of tissue, called the plantar fascia, that runs across the bottom of the foot and connects the heel bone to the toes. For many individuals, plantar fasciitis may be treated conservatively with anti-inflammatory drugs and stretching and strengthening exercises. Where conservative treatment does not resolve the plantar fasciitis, surgical treatment may be necessary.

### BRIEF DESCRIPTION OF THE DRAWINGS

Non-limiting and non-exhaustive examples of the present invention are described with reference to the following figures, wherein like reference numerals refer to like parts throughout the various views unless otherwise specified.

FIG. 1A shows a drawing overview of a method and system for treatment of plantar fasciitis using a clamping arthroscopic resection tool.

FIG. 1B shows a drawing overview of a method and system for treatment of plantar fasciitis using a clamping arthroscopic resection tool.

FIG. 1C shows a drawing overview of a method and system for treatment of plantar fasciitis using a clamping arthroscopic resection tool.

FIG. 2 shows a side view of an embodiment of the clamping arthroscopic resection tool.

FIG. 3 shows a perspective view of an embodiment of the clamping arthroscopic resection tool.

FIG. 4A shows a bottom view of an embodiment of the clamping arthroscopic resection tool.

FIG. 4B shows a top view of an embodiment of the clamping arthroscopic resection tool.

FIG. 5 shows a disassembled perspective view of an embodiment of the clamping arthroscopic resection tool.

FIG. 6A shows a drawing of the clamping arthroscopic resection tool used during surgery.

FIG. 6B shows a drawing of the clamping arthroscopic resection tool used during surgery.

FIG. 6C shows a drawing of the clamping arthroscopic resection tool used during surgery.

FIG. 6D shows a drawing of the clamping arthroscopic resection tool used during surgery.

FIG. 6E shows a drawing of the clamping arthroscopic resection tool used during surgery.

FIG. 7A shows a bottom view drawing of the second body member of the clamping arthroscopic resection tool.

FIG. 7B shows a side view drawing of the second body member of the clamping arthroscopic resection tool.

FIG. 7C shows a top view drawing of the second body member of the clamping arthroscopic resection tool.

FIG. 8A shows a bottom view drawing of the first body member of the clamping arthroscopic resection tool.

FIG. 8B shows a side view drawing of the first body member of the clamping arthroscopic resection tool.

FIG. 8C shows a top view drawing of the first body member of the clamping arthroscopic resection tool.

FIG. 9A shows a side view drawing of the third body member, the cutting member, of the clamping arthroscopic resection tool.

FIG. 9B shows a bottom view drawing of the third body member, the cutting member, of the clamping arthroscopic resection tool.

FIG. 10 shows a side view of a disposable clamping arthroscopic resection tool.

FIG. 11 shows a perspective view of a disposable clamping arthroscopic resection tool.

FIG. 12 shows a perspective view of a portion of a disposable clamping arthroscopic resection tool.

FIG. 13A shows a top view of a disposable clamping arthroscopic resection tool.

FIG. 13B shows a bottom view of a disposable clamping arthroscopic resection tool.

FIG. 14 shows a disassembled perspective view of a disposable clamping arthroscopic resection tool.

FIG. 15A shows a drawing of the disposable clamping arthroscopic resection tool used during surgery.

FIG. 15B shows a drawing of the disposable clamping arthroscopic resection tool used during surgery.

FIG. 15C shows a drawing of the disposable clamping arthroscopic resection tool used during surgery.

FIG. 15D shows a drawing of the disposable clamping arthroscopic resection tool used during surgery.

FIG. 15E shows a drawing of the disposable clamping arthroscopic resection tool used during surgery.

FIG. 16 shows a perspective view of the left half of the body of the disposable clamping arthroscopic resection tool.

FIG. 17A shows a top view of the lower jaw of the disposable clamping arthroscopic resection tool.

FIG. 17B shows a side view of the lower jaw of the disposable clamping arthroscopic resection tool.

FIG. 17C shows a perspective view of the lower jaw of the disposable clamping arthroscopic resection tool.

FIG. 17D shows a sheet metal cutout view of the lower jaw of the disposable clamping arthroscopic resection tool.

FIG. 18A shows a side view of the clamp actuation button and associated pushrod of the disposable clamping arthroscopic resection tool.

FIG. 18B shows a bottom view of the clamp actuation button and associated pushrod of the disposable clamping arthroscopic resection tool.

FIG. 18C shows a perspective view of the clamp actuation button and associated pushrod of the disposable clamping arthroscopic resection tool.

FIG. 18D shows a top view of the clamp actuation button pushrod of the disposable clamping arthroscopic resection tool.

FIG. 19A shows a perspective view of the cutting member of the disposable clamping arthroscopic resection tool.

FIG. 19B shows a side view of the cutting member of the disposable clamping arthroscopic resection tool.

FIG. 19C shows a top view of the cutting member of the disposable clamping arthroscopic resection tool.

Corresponding reference characters indicate corresponding components throughout the several views of the drawings. Skilled artisans will appreciate that elements in some of the figures are illustrated for simplicity and clarity. The figures have been drawn to scale in showing the arthroscopic resection tool. As such, the figures allow for visualization and comparison of the sizes and dimensions of some of the elements in the figures relative to other elements to help to improve understanding of various examples of the present invention. In some figures, some elements are not depicted in order to facilitate a less obstructed view of other elements.

It will be appreciated that the drawings are illustrative and not limiting of the scope of the invention which is defined by the appended claims. The examples shown each accomplish various different advantages. It is appreciated that it is not possible to clearly show each element or advantage in a single figure, and as such, multiple figures are presented to separately illustrate the various details of the examples in greater clarity. Similarly, not every example need accomplish all advantages of the present disclosure.

### DETAILED DESCRIPTION

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

Reference throughout this specification to “one embodiment”, “an embodiment”, “one example” or “an example” means that a particular feature, structure or characteristic described in connection with the embodiment or example is included in at least one embodiment of the present invention. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combination and/or sub-combinations in one or more embodiments or examples. In addition, it is appreciated that the figures provided herewith are for explanation purposes to persons ordinarily skilled in the art and that the drawings are not necessarily drawn to scale.

The disclosure particularly describes a device and method for performing a partial release of soft tissue such as the plantar fascia ligament. Plantar fasciitis which is not resolved through conservative treatment may require surgical treatment. A partial release of a tissue is where a tissue such as a ligament or tendon is partially cut to release tension in the tissue. During partial release surgery to treat plantar fasciitis, the plantar fascia ligament is partially cut to relieve tension in the ligament and provide relief of the plantar fasciitis pain. A difficulty in partially releasing the plantar fascia ligament is in cutting the ligament smoothly and to an appropriate depth. The plantar fascia ligament may be cut from the side to remove a portion of the ligament. If the ligament is over cut, stretching or failure of the ligament may occur. Overcutting of the ligament may cause tearing of the ligament or a loss of support in the arch and chronic pain which is not easily resolved. If the ligament is cut in a manner which leaves a rough surface or spurs on the tendon, the condition of the ligament may result in residual pain or create locations where the ligament may be weakened and tear.

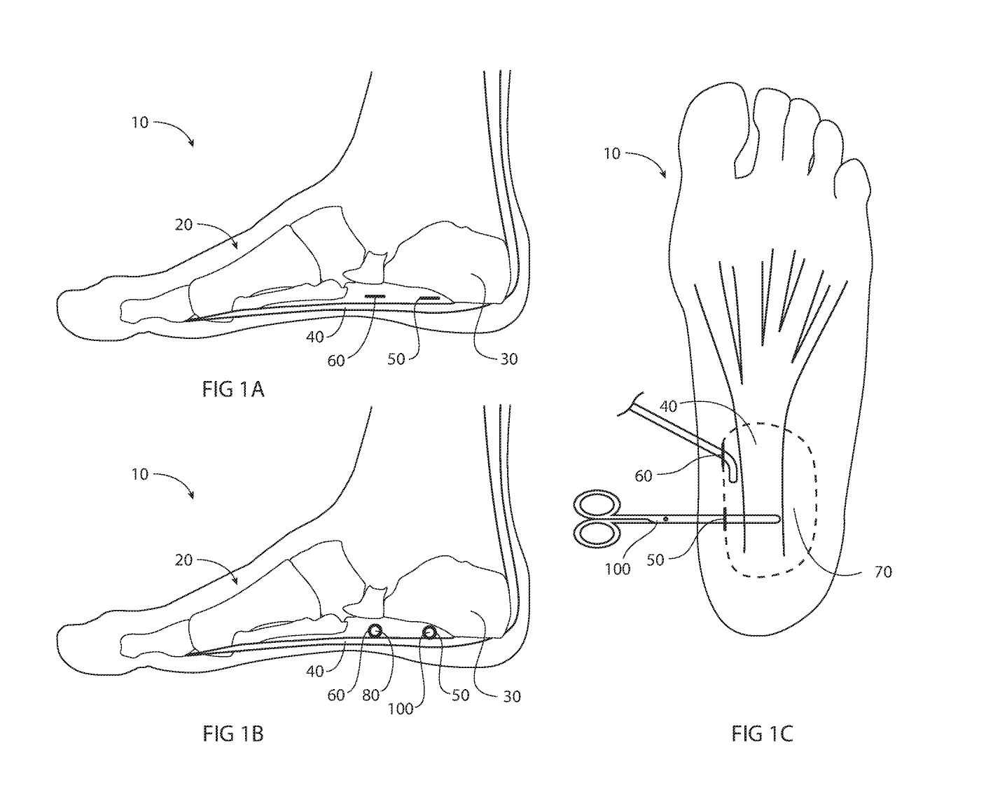

FIGS. 1A and 1B show an overview of a method and system for treatment of plantar fasciitis using a clamping arthroscopic resection tool 100 of the present invention. FIGS. 1A and 1B show side views of a foot while FIG. 1C shows a top view of the foot and the clamping arthroscopic resection tool 100. The foot 10 is drawn with major foot bones 20 and with the calcaneus 30 (heel bone) specifically. The plantar fascia 40 is a thick and strong band of connective tissue that attaches to the calcaneus 30 and extends along the sole of the foot 10 before connecting at the base of the toes. The plantar fascia 40 branches near the middle of the foot and is attached to each toe. The plantar fascia supports the arch of the foot. If a patient is afflicted by plantar fasciitis, the plantar fascia 40 is inflamed; causing the patient pain. The plantar fascia 40 may be partially released to reduce tension in the plantar fascia 40 and help reduce chronic inflammation. In order to partially release the plantar fascia 40, a small first incision 50 (approximately 1 cm long) is made slightly proximal (between the body and the plantar fascia 40) and anterior to the plantar fascia origin at the calcaneus 30. A second incision 60 (approximately 1 cm long) is made proximal to the plantar fascia 40 and approximately 2 cm anterior to the first incision 50. An 18 gauge spinal needle may be used to help determine the location of a second incision. FIG. 1A shows the first incision 50 and the second incision 60.

As is illustrated in FIG. 1B, an arthroscope 80 is introduced into the plantar fascia space through the second incision 60. The arthroscope 80 is used to introduce saline into the plantar fascia space at low pressure to create an operative space 70 within the tissue surrounding the plantar fascia 40. During the surgery, the arthroscope 80 typically remains in the second opening 60 and other tools are introduced into the operative space 70 through the first incision 50. A shaver may be introduced into the operative space 70 through the first incision 50 and then used to debride any subcalcaneal bursa tissue to allow better visualization of the plantar fascia 40.

The plantar fascia 40 is inspected to identify any fraying or tearing of the plantar fascia 40. Fraying of the plantar fascia 40 is similar to an unraveling rope and presents poorly organized tissue. Plantar fascia tears are typically interstitial, longitudinal tears which are in line with the long axis of the plantar fascia 40. The plantar fascia 40 is debrided until stable, organized tissue remains. Once the plantar fascia debridement is complete, the partial plantar fascia release may be performed with a clamping arthroscopic resection tool 100. The clamping arthroscopic resection tool 100 is inserted into the operative space 70 through the incision 50. FIGS. 1B and 1C show the arthroscope 80 and the clamping arthroscopic resection tool 100 inserted into incisions 60 and 50 respectively. The goal of the release is often to release ⅓ to ½ of the plantar fascia 40. In other words, the goal of the surgical release is often to remove approximately ⅓ to ½ of the lateral width of the plantar fascia 40 in the area of the incision 50; cutting completely through the vertical thickness of the plantar fascia 40.

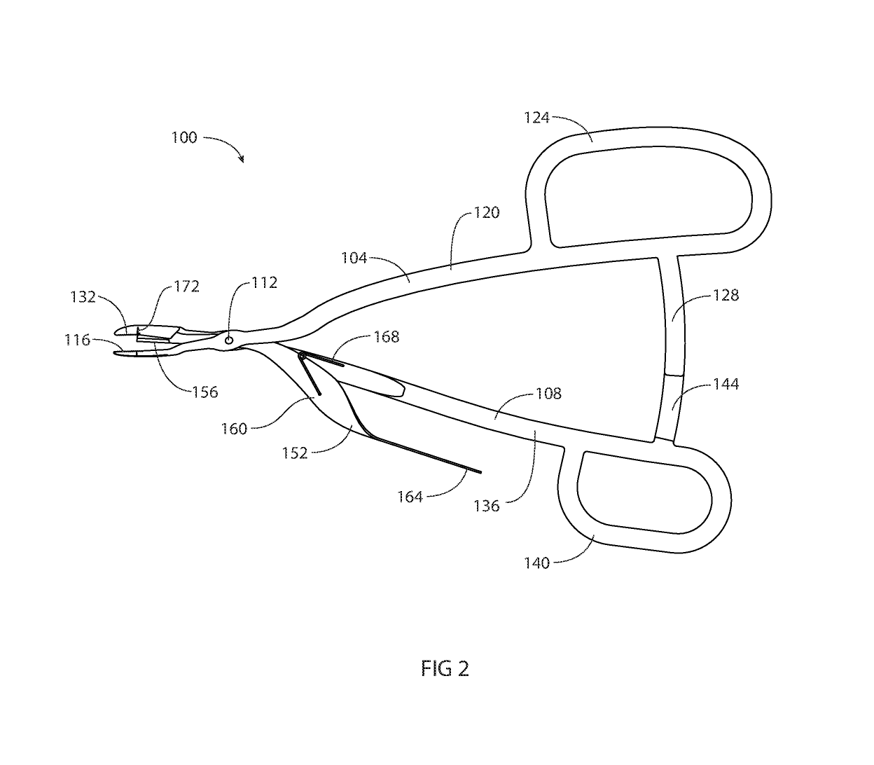

As can be seen in FIG. 1C, the clamping arthroscopic resection tool 100 includes clamping jaws which may extend across all of or across a large portion of the plantar fascia 40. The clamping jaws extend farther across the plantar fascia 40 than the cutting blade will cut. This allows the surgeon to securely hold the plantar fascia 40 both before and after resection of the plantar fascia. As shown in FIG. 1C and illustrated in FIGS. 2 through 5, the clamping jaws of the clamping arthroscopic resection tool 100 may extend laterally across and hold approximately ½ to all of the lateral width of the plantar fascia 40 during resection of the plantar fascia 40. More preferably, the clamping arthroscopic resection tool 100 may extend laterally across and hold approximately ⅔ to all of the lateral width of the plantar fascia 40, or alternatively approximately ⅔ to ¾ of the lateral width of the plantar fascia 40, during resection of the plantar fascia 40. The clamping plantar fascia resection tool 100 includes clamping jaws which extend distally farther than its cutting blade to allow a surgeon to securely hold the plantar fascia 40 during the resection of the plantar fascia 40 and maintain precise control over the resection.

Additional debridement with a shaver may be required at this time to make sure that stable, organized plantar fascia fibers remain. The plantar fascia 40 should be inspected as far proximally as possible to ensure there is no impingement of the calcaneus 30 on the plantar fascia 40 and, if necessary, calcaneal exostectomy may be performed with a shaver or a burr. The plantar fascia should also be visualized as far distally as possible to ensure the integrity of the entire plantar fascia. Once any debridement is complete, the operative space 70 is irrigated to avoid leaving any debris behind. The incisions 50, 60 are closed with suture and the wounds are dressed and cared for.

During surgery, it is important to properly cut the plantar fascia 40. Remaining fibrous spurs can cause problems such as irritation or inflammation of the plantar fascia and surrounding tissues or may provide weakened locations where the plantar fascia 40 may tear. An overcut plantar fascia 40 may provide insufficient strength and can continue to tear little by little and, over time leave a patient without an arch in the foot. An overcut plantar fascia 40 may not be strong enough and may then present chronic pain and inflammation. The clamping arthroscopic resection tool 100 assists with many of these difficulties. The clamping arthroscopic resection tool 100 allows for better visualization of the entire width of the plantar fascia 40 and for controlled cutting of the plantar fascia 40. The clamping arthroscopic resection tool 100 clamps and holds the plantar fascia 40 while cutting and cuts perpendicularly through the plantar fascia 40 at a predetermined depth. The clamping arthroscopic resection tool 100 provides for safe and repeatable release of the plantar fascia, gastrocnemius, carpal tunnel, and for other soft tissue releases.

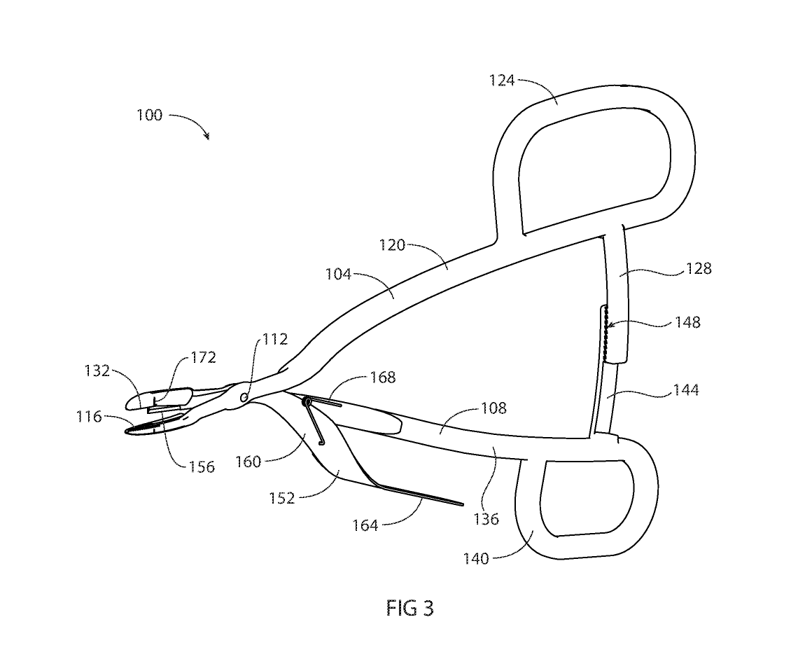

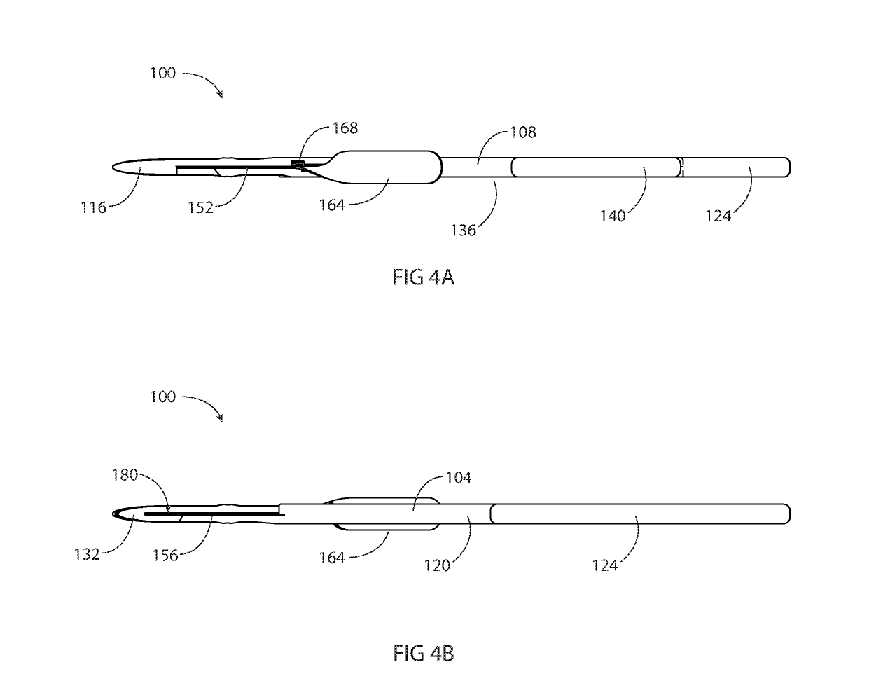

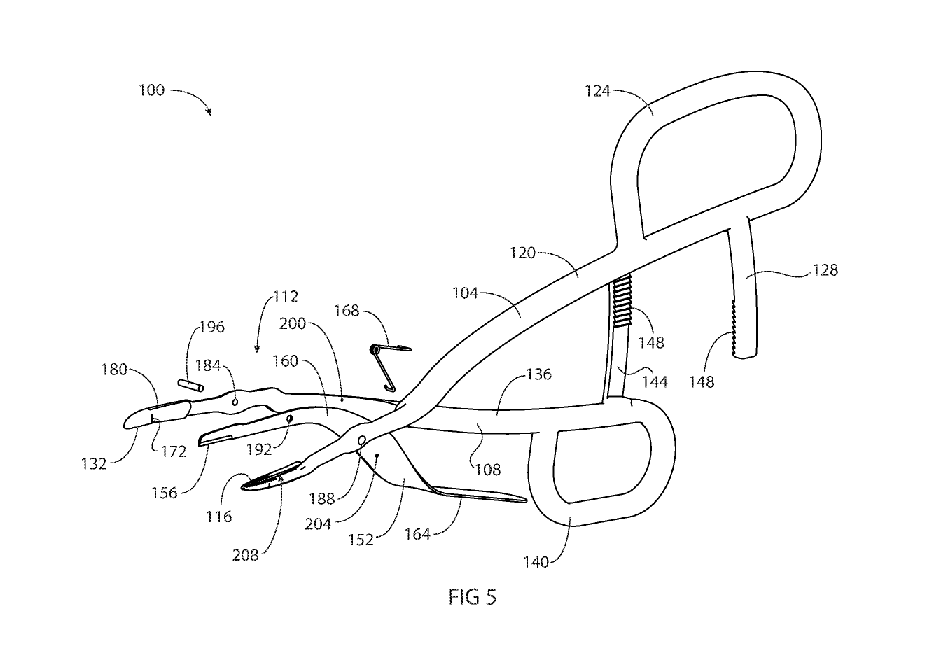

FIGS. 2 through 5 show different views of an embodiment of the clamping arthroscopic resection tool 100. FIG. 2 shows a side view of the clamping arthroscopic resection tool 100. FIG. 3 shows a perspective view of the clamping arthroscopic resection tool 100. FIGS. 4A and 4B show bottom and top views of the clamping arthroscopic resection tool 100. FIG. 5 shows a disassembled view of the clamping arthroscopic resection tool 100.

The clamping arthroscopic resection tool 100 includes a first body member 104 and a second body member 108 which are pivotably attached to each other at a pivot 112. The first body member 104 includes a serrated jaw 116, a shank 120, and a handle such as a finger ring 124. The serrated jaw 116 is disposed distally from the pivot 112 and the shank 120 and finger ring 124 are disposed proximally from the pivot 112. The first body member also includes a ratcheting clamp member 128 which extends from the shank 120 or finger ring 124 towards the second body member 108. The second body member 108 also includes a serrated jaw 132, a shank 136, and a handle such as a finger ring 140. The serrated jaw 132 is disposed distally from the pivot 112 and the shank 136 and finger ring 140 are disposed proximally from the pivot 112. The second body member also includes a ratcheting clamp member 144 which extends from the shank 136 or finger ring 140 towards the first body member 104. The first serrated jaw 116 and the second serrated jaw 132 are disposed adjacent each other and the serrated surfaces of these jaws face each other to allow the arthroscopic resection tool 100 to clamp tissue between the serrated jaws. The finger rings 124, 140 allow a person to place their fingers therein during use of the clamping arthroscopic resection tool 100; allowing the person to both guide and move the tool 100 as well as to open and close the serrated jaws 116, 132.

The first body member 104 and the second body member 108 rotate with respect to each other about the pivot 112, allowing the jaws 116, 132 to be brought towards or away from each other by moving the finger rings 124, 140 towards each other or away from each other. When the serrated jaws 116, 132 are brought in relatively close proximity to each other, the ratcheting clamp members 128, 144 engage each other; allowing complementary shaped ratcheting teeth 148 formed on both the first clamp member 128 and the second clamp member 144 to engage each other and prevent the jaws 116, 132 from moving away from each other until an additional clamping force and/or lateral pressure is applied to the finger rings 124, 140 to unlock the jaws 116, 132 by moving the ratcheting clamp members 128, 144 away from each other. In this manner, the clamping arthroscopic resection tool 100 may be used to grip a tissue such as the plantar fascia 40 and lock onto the plantar fascia to hold it securely. The clamping arthroscopic resection tool 100 is designed such that the jaws 116, 132 may clamp onto tissues which are between 0 mm and 10 mm thick, and more preferably tissues between 2 mm and 5 mm thick while the finger bows 124, 140 remain in a comfortable position and the clamp members 128, 144 engage each other to clamp the clamping arthroscopic resection tool 100 onto the target tissue.

The clamping arthroscopic resection tool 100 includes a third body member 152 which forms a cutting attachment. The cutting attachment 152 is pivotably attached to the first body member 104 and second body member 108 and may be attached to the first body member 104 and second body member 108 at the pivot 112 and pivot about the pivot 112. The cutting attachment 152 includes a cutting blade 156 which extends distally away from the pivot 112 and which is disposed between the first jaw 116 and second jaw 132. The cutting attachment also includes a shank 160 and a finger grip 164. The shank 160 and the finger grip 164 extend proximally away from the cutting blade 156 and proximally away from the pivot 112. The shank 160 and the finger grip 164 extend generally along the shank 136 of the second body member 108. A spring 168, or other suitable biasing element, is attached to the second body member 108 and to the cutting attachment 152 to bias the cutting attachment finger grip 164 away from the second body member shank 136. The spring 168 pushes the shank 160 of the cutting attachment 152 away from the shank 136 of the second body member 108.

The biasing of the spring 168 moving the finger grip 164 away from the shank 136 and thereby moves the blade 156 away from the first jaw 116 to a position adjacent the second jaw 132 where the blade 156 does not extend past the gripping surface of the second jaw 132 and will not cut tissue placed between the first and second jaws 116, 132. The blade 156 may be disposed in a slot formed in the second serrated jaw 132 or may be located next to the serrated jaw 132. A stop may be formed on the second serrated jaw 132 or on the shank 136 which stops the movement of the cutting attachment 152 against the biasing of the spring 168 and keeps the cutting attachment in a retracted position unless moved by a person.

The spring 168 allows a person to place the clamping arthroscopic resection tool 100 so that a target tissue is positioned between the first jaw 116 and second jaw 132 and clamp the clamping arthroscopic resection tool 100 onto the tissue so that the tissue is held securely between the first jaw 116 and the second jaw 132 without cutting the tissue. Movement of the finger grip 164 towards the shank 136 of the second body member 108 will move the blade 156 past the clamping surface of the second jaw 132, through tissue positioned between the first jaw 116 and second jaw 132 and adjacent the first jaw 116. Releasing the finger grip 164 will allow the spring 168 to retract the blade 156 from adjacent the first jaw 116 to its resting position adjacent the second jaw 132. The finger grip 164 extends proximally towards the second finger bow 140 and stops short of the second finger bow 140 to leave a space between the finger grip 164 and the second finger bow 140. This allows a surgeon to manipulate the clamping arthroscopic resection tool 100 within the operative cavity 70 and clamp the tool 100 onto the plantar fascia 40 without contacting the finger grip 164 and operating the blade 156. The design also allows the surgeon to operate the finger grip 164 and blade 156 while still maintaining control of the clamping arthroscopic resection tool 100.

The second jaw 132 includes an indicator mark 172 along the side of the second jaw 132 at a position adjacent the tip of the cutting blade 156. If desired, the first jaw 116 may also include a correspondingly placed indicator mark 176 on the side of the first jaw 116 at the location where the tip of the cutting blade 156 meets the first jaw 116 when used to cut tissue. The indicator mark 172 shows how far the blade 156 extends relative to the length of the jaws 116, 132 and shows the distal extent to which the blade 156 will cut tissue during surgery. The indicator mark 172 is particularly useful when used in combination with an arthroscope during arthroscopic surgery as the indicator mark can be seen from the arthroscope 80.

The clamping arthroscopic resection tool 100 allows the surgeon to hold and position the plantar fascia 40 with the jaws 116, 132 and allows the surgeon to visualize the size and shape of the plantar fascia 40. The surgeon is able to hold the plantar fascia 40 between the jaws 116, 132 and, using the indicator mark 172, visualize the fraction of the plantar fascia which will be cut by the blade 156 according to the present position of the clamping arthroscopic resection tool 100 relative to the plantar fascia 40. The plantar fascia 40 does not move relative to the jaws 116, 132 or to the blade 156 while the plantar fascia is cut so the cut is precisely as determined by the surgeon while clamping the plantar fascia between the jaws 116, 132.

Referring to FIGS. 4A and 4B, bottom and top views of the clamping arthroscopic resection tool 100 are shown. It can be seen how the cutting attachment 152 extends generally parallel to the shank 136 of the second body member 108. The cutting attachment 152 may be bent between the shank 160 and the finger grip 164 to provide a more comfortable finger grip 164. The cutting blade 156 may be centered laterally relative to the width of the clamping arthroscopic resection tool 100. As seen, the cutting blade 152 may be placed between the first body member 104 and the second body member 108 and these three components may be held together by a pin at the pivot 112. The second jaw 132 may be formed with a slot 180 which extends through the thickness of the second jaw 132 and which receives the cutting blade 156 therethrough. The slot 180 allows the cutting blade 156 to pass through the center of the proximal portion of the second jaw 132. This allows the sharpened cutting edge of the cutting blade 156 to be located beneath the clamping surface of the second jaw 132 in a retracted position. The spring 168 biases the cutting blade 156 into the retracted position. When the finger grip 164 is pressed, the sharpened edge of the cutting blade 156 is extended beyond the clamping surface of the second jaw 132 and passes through a tissue held between the first jaw 116 and the second jaw 132. The first jaw 116 may have a small recess or a slot 208 (FIG. 5) formed in its clamping surface to allow the cutting blade 156 to pass completely through the tissue and make a complete cut.

FIG. 5 shows a disassembled view of the clamping arthroscopic resection tool 100. The pivot 112 is formed by a hole 184 formed in the first body member 104, a hole 188 formed in the second body member 108, a hole 192 formed in the third body member 152, and a pin 196. The hinge 112 could be formed from alternate structures such as a bolt which passes through these holes 184, 188, 192, or a post formed in place of a hole on one of the body members 104, 108, 152. It will be appreciated that the third body member (cutting attachment) 152 need not pivot about the same axis or pivot point as the first body member 104 and second body member 108, although this is convenient. The second body member 108 may include a hole 200 or other attachment structure which allows the spring 168 to attach to the second body member. The third body member 152 may similarly include a hole 204 for attachment to the spring 168. Alternatively, the second body member 108 and the third body member 152 may each have a post which engages a coil compression spring located between the second body member 108 and the third body member 152 to push these apart and bias the cutting blade 156 into a retracted position. These structures bias the third body member 152 relative to the second body member 108 so that the cutting blade 156 is held in a retracted, non-cutting position. In this non-cutting position, the cutting blade 156 may be located in the slot 180 formed in the second jaw 132. As seen, the second jaw 132 may define a generally U shaped structure extending forwards towards the distal end of the clamping arthroscopic resection tool 100, laterally in front of the slot 180, and backwards proximally. This shape encloses the cutting blade 156 in the second jaw 132 and protects the cutting blade 156 from damage as well as protecting tissue from inadvertent cutting. As seen, the cutting blade 156 may extend along a proximal portion of the first jaw 116 and second jaw 132. The first jaw 116 and second jaw 132 also have distal portions which extend distally beyond the cutting blade 156. Accordingly, the cutting blade 156 may extend along approximately ½ of the clamping jaws 116, 132, or along between approximately ⅓ and ⅔ of the clamping jaws. Because the distal portions of the clamping jaws 116, 132 extend beyond the cutting blade 152, the distal portions of the clamping jaws 116, 132 remain attached to the plantar fascia 40 when it is cut.

FIG. 5 also illustrates how the first jaw 116 may also include a slot or channel 208 which receives the cutting blade 156 as it is moved into a cutting position. The slot 208 in the first jaw 116 allows the cutting blade 156 to pass completely through and beyond the target tissue to cut completely through the tissue. The first jaw 116 may define a generally U shaped structure extending forwards towards the distal end of the clamping arthroscopic resection tool 100, laterally in front of the slot 208, and backwards proximally. This shape allows the cutting blade 156 to bypass the first jaw 116 and pass completely through the tissue being cut rather than using the first jaw 116 as an anvil and cutting the tissue against the first jaw 116. This blade and jaw design produces a clean cut in the target tissue.

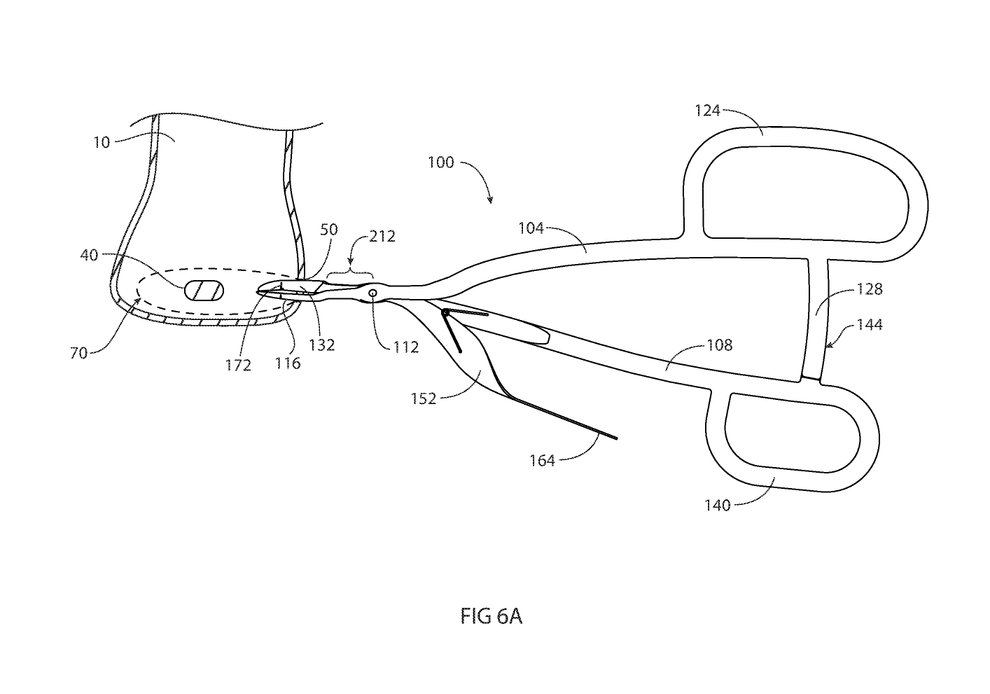

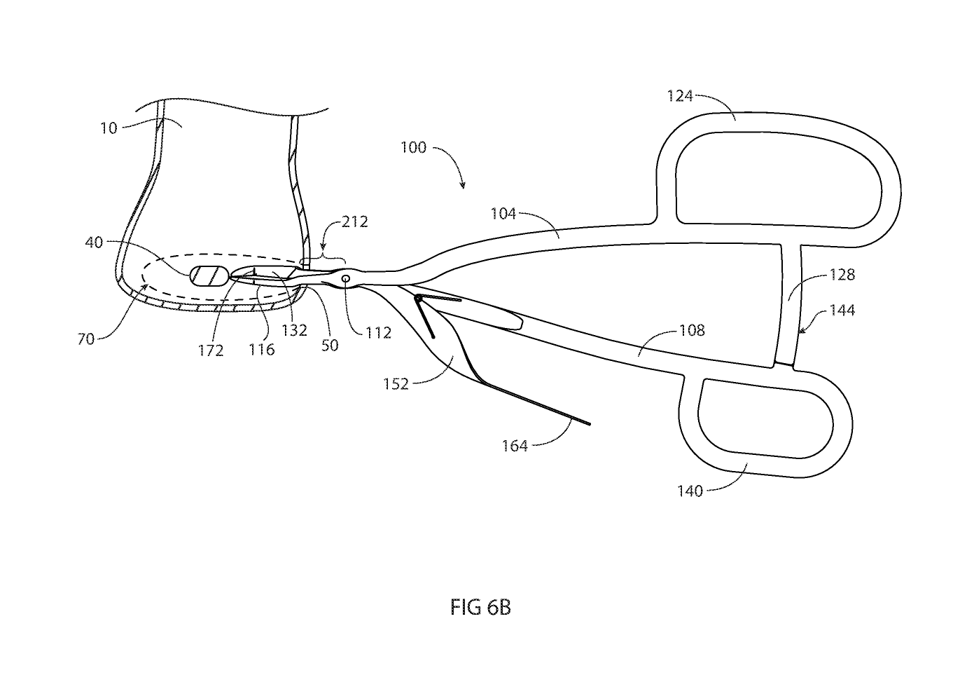

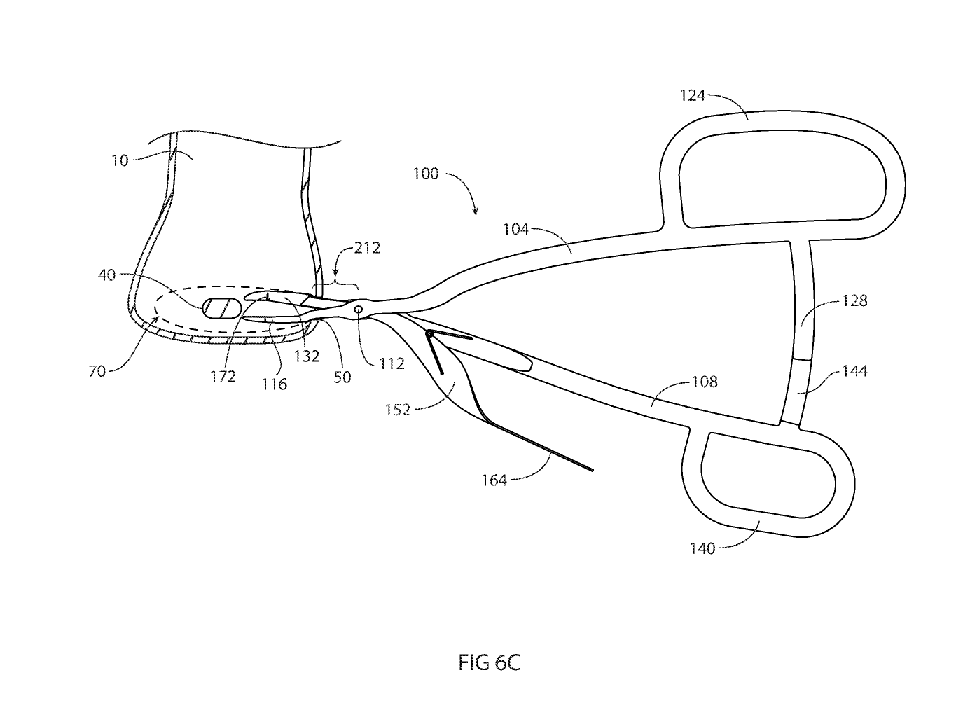

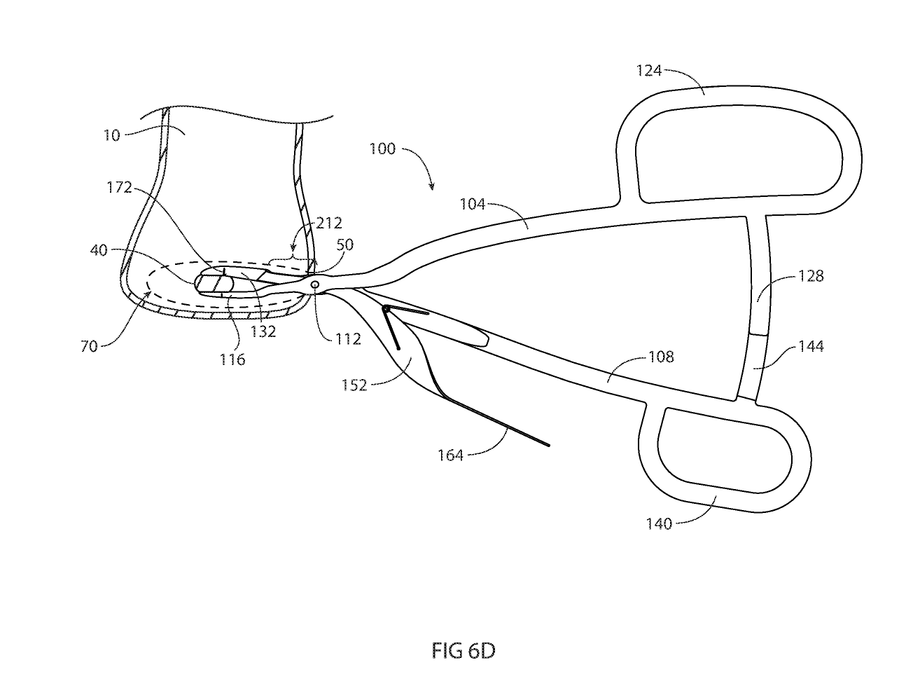

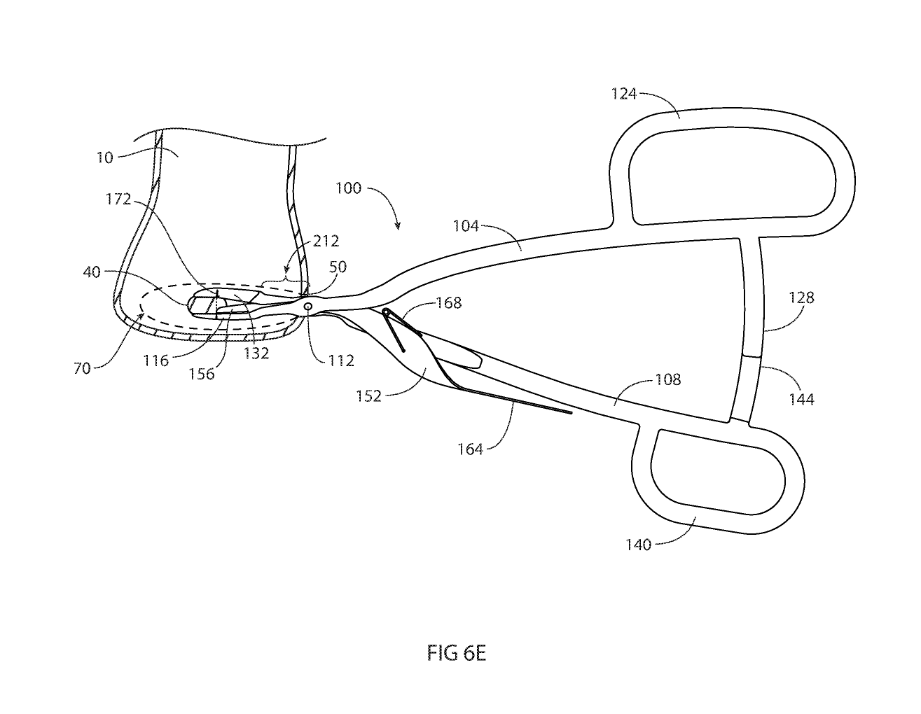

FIGS. 6A through 6E show drawings of the clamping arthroscopic resection tool 100 used during surgery. These drawings also show a cross section of the foot 10 during surgery. These drawings show a view of the operative space 70 and of the clamping arthroscopic resection tool 100 which is similar to what a surgeon may see from the arthroscope 80 during surgery. As discussed above, the foot 10 has been prepared for surgery and a first incision 50 and a second incision 60 have been made in the foot. A 1 cm incision will allow insertion of a 6 mm to 6.5 mm diameter tool, and the clamping arthroscopic resection tool 100 may be sized accordingly if it is desired to minimize the size of the incision 50. The arthroscope 80 has been placed into the operative space 70 through the second incision 60 and the operative space 70 has been created by inflation with saline. Any initial debridement or preparation of the calcaneus 30 and/or the plantar fascia 40 has been performed. The clamping arthroscopic resection tool 100 is introduced into the operative space 70 through the first incision 50. During insertion of the clamping arthroscopic resection tool 100, the first jaw 116 and second jaw 132 may be clamped against each other and the clamp members 128 and 144 may be locked together. The first jaw 116 or first body member 104 may include a pin or wall which prevents the blade 156 from extending past the outer surface of the first jaw 116 in order to minimize any risk of accidental cutting while inserting or manipulating the clamping arthroscopic resection too. 100.

The clamping arthroscopic resection tool 100 may be maneuvered within the operative space 70 into a desired position relative to the plantar fascia 40 as shown in FIG. 6B. The surgeon will position the clamping arthroscopic resection tool 100 at a desired length along the length of the plantar fascia 40. The surgeon may release the engagement of the first clamp member 128 and second clamp member 144 and open the jaws 116, 132 of the clamping arthroscopic resection tool 100 as shown in FIG. 6C by moving the first finger loop 124 and the second finger loop 140 away from each other. The surgeon may then place the first jaw 116 and second jaw 132 of the clamping arthroscopic resection tool 100 on opposing sides of the plantar fascia 40 as shown in FIG. 6D and maneuver the clamping arthroscopic resection tool 100 into a desired position relative to the plantar fascia 40. The clamping arthroscopic resection tool 100 is placed in a position where it crosses the plantar fascia 40 transversely as is also shown in FIG. 1B.

Using the arthroscope 80, the surgeon can visualize the size of the plantar fascia 40 well using the jaws 116, 132 of the clamping arthroscopic resection tool 100. The indicator mark 172 on clamping jaw 132 (as well as a corresponding indicator mark on clamping jaw 116 if provided) may be used by the surgeon to assess the depth of cut of the clamping arthroscopic resection tool 100 and to visually divide the plantar fascia 40 into a cut portion and an uncut portion while conducting the surgery. The indicator mark 172 allows the surgeon to precisely assess how much of the plantar fascia 40 will remain uncut and how much of the plantar fascia will be cut during surgery. The clamping arthroscopic resection tool 100 thus allows a surgeon to precisely evaluate the size of the plantar fascia 40 and to determine an amount of the plantar fascia 40 to resect.

Once the clamping arthroscopic resection tool 100 is placed in a desired position across the plantar fascia 40, the surgeon may clamp the clamping arthroscopic resection tool 100 onto the plantar fascia 40 by applying pressure to the finger bows 124, 140. The first ratcheting clamp member 128 and the second ratcheting clamp member 144 engage each other and the clamping arthroscopic resection tool 100 is securely attached to the plantar fascia 40. The surgeon may easily control the pressure applied to the plantar fascia by the clamping arthroscopic resection tool 100. The first clamp member 128 and the second clamp member 144 engage each other and prevent loosening of the clamping arthroscopic resection tool 100 unless released by the surgeon. Once the clamping arthroscopic resection tool 100 is clamped onto the plantar fascia 40, the depth of cut through the side of the plantar fascia 40 is fixed and does not change as the surgeon completes the release.

As shown in FIG. 6E, the surgeon may then apply pressure to the finger grip 164 of the third body member 152, moving the blade 156 through the plantar fascia 40 so that the distal end of the blade 156 passes through the plantar fascia 40 at the indicator mark 172. The cut through the plantar fascia 40 is made at the precise depth indicated by the indicator mark 172 as previously determined by the surgeon's positioning of the clamping arthroscopic resection tool 100. The cut through the plantar fascia 40 is also made perpendicularly through the plantar fascia 40 and the plantar fascia 40 is cut evenly with the blade 156 cutting through the plantar fascia 40 at the same depth from the top to the bottom of the plantar fascia. After cutting the plantar fascia 40, the surgeon may release pressure on the finger grip 164 and allow the cutting blade 156 to move to a retracted position. The surgeon may then unlock the first clamp member 128 and second clamp member 144 to release the clamping arthroscopic resection tool 100 from the plantar fascia 40. The surgeon may, in like manner, make any other necessary cuts in the plantar fascia 40 and may remove the clamping arthroscopic resection tool 100 from the operative space 70 and from the foot 10.

In viewing FIGS. 6A through 6E, it can be seen how the clamping arthroscopic resection tool 100 may be made with a neck 212 located proximally of the jaws 116, 132 which is a reduced size compared to the jaws 116, 132. The reduced size neck 212 is located between the clamping jaws 16, 132 and the pivot 112. The reduced size of the neck 212 allows the clamping arthroscopic resection tool 100 to be more easily maneuvered through a small incision 50. Similarly, the clamping arthroscopic resection tool 100 is made relatively small at the pivot 112 to allow for use in through a minimal incision 50. The clamping arthroscopic resection tool 100 may be constructed such that the indicator mark 172 and distal edge of the cutting blade 156 are located between approximately 2.5 cm and 3 cm from the pivot 112. This sizing allows the jaws 116, 132 to be opened and maneuvered within the operative space 70 created within the foot 10 while reduced neck 212 and the pivot 112 are at or near the incision 50 and allows for operation of the clamping arthroscopic resection tool 100 through a small incision 50.

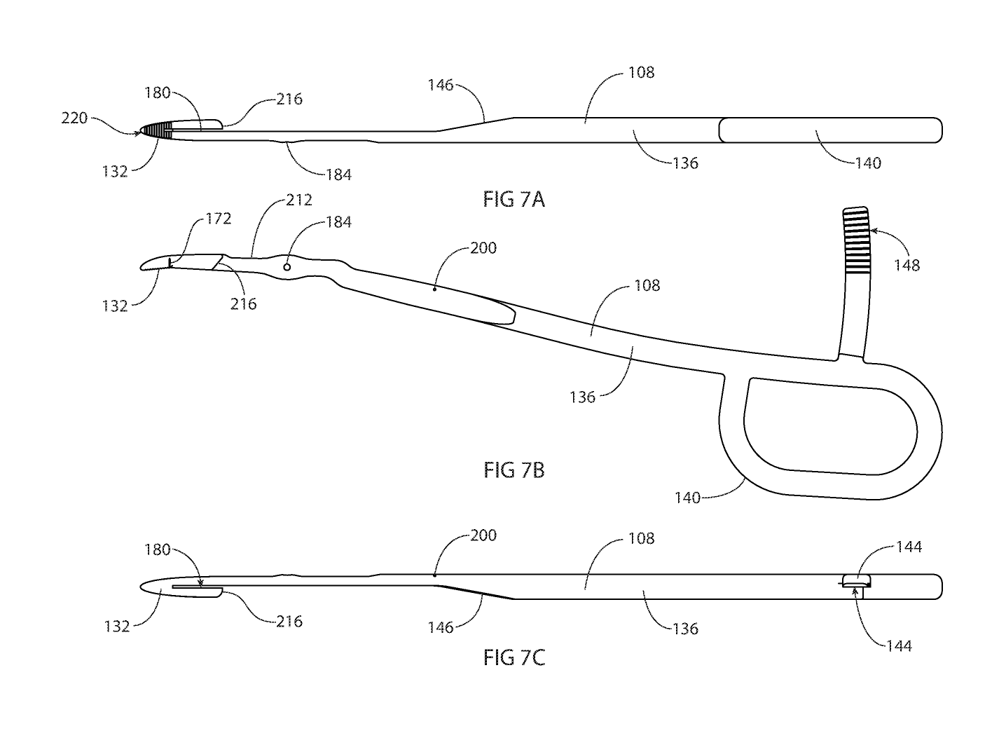

FIGS. 7A through 7C show bottom, side, and top view drawings of the second body member 108. These figures illustrate how the slot 180 is positioned such that it extends through a proximal and central position on the second jaw 132. The slot 180 is aligned with the central axis of the clamping arthroscopic resection tool 100. From the neck 212, the second jaw 132 extends distally towards the tip of the clamping arthroscopic resection tool 100, laterally, and then proximally away from the tip of the clamping arthroscopic resection tool 100 in a U or V shape. This may result in the jaw 132 having a freestanding side 216. Also visible are serrations 220 formed on the surface of the second jaw 132 to aid in gripping the tissue.

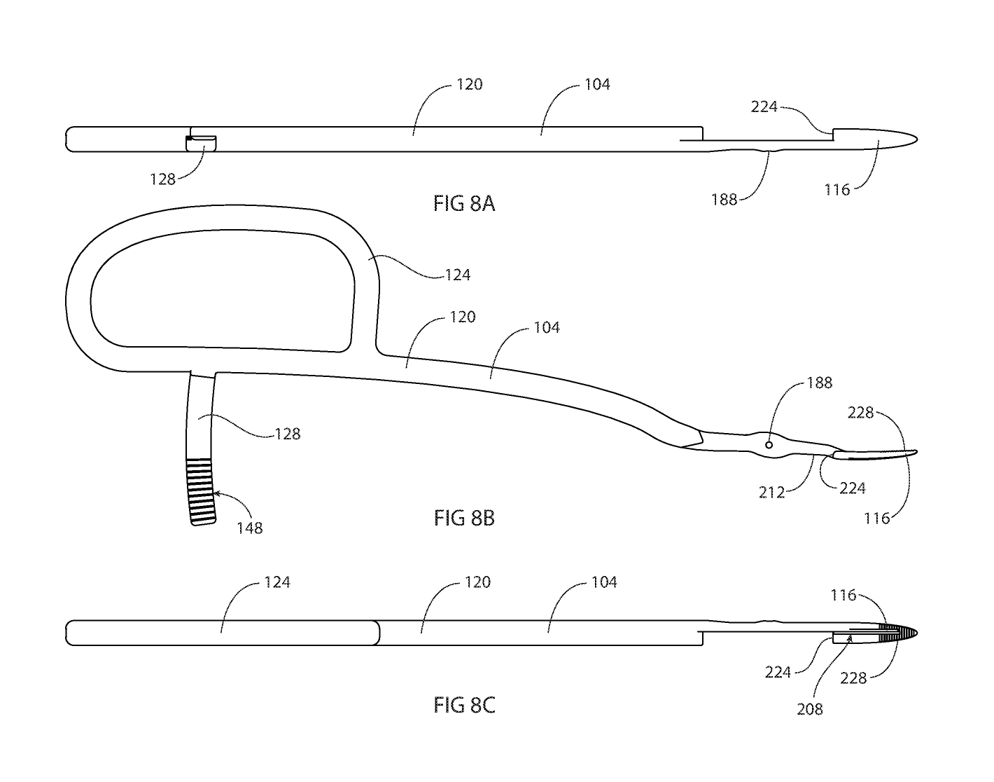

FIGS. 8A through 8C show bottom, side, and top view drawings of the first body member 104. These figures illustrate how the channel/slot 208 is formed in the first jaw 116. The slot 208 is positioned such that it extends into a proximal and central position on the first jaw 116. The slot 208 may be formed so that it extends downwardly through the first jaw 116. Alternatively, the slot 208 may be formed so that it extends downwardly into the first jaw 116 through the clamping surface of the jaw 116 but does not extend completely through the thickness of the first jaw 116. This provides several benefits. Forming the first jaw 116 so that the slot 208 does not extend through the jaw 116 strengthens the jaw 116 as compared to extending a slot completely through the jaw; allowing the jaw 116 to be made thinner while retaining sufficient strength. Additionally, the blade 156 is prevented from passing through the jaw 116 since the slot 208 does not pass completely through the jaw 116. This prevents accidental damage to tissue which may be adjacent to the outside of the jaw 116 while using the clamping arthroscopic resection tool 100 to perform a desired cut in tissue.

The slot 208 is aligned with the central axis of the clamping arthroscopic resection tool 100 and is aligned laterally as well as distally with the slot 180 in the second jaw 132 so that the blade 156 may pass without obstruction from a non-cutting position in the slot 180, through tissue such as the plantar fascia 40, and into the slot 208. From the neck 212, the first jaw 116 extends distally towards the tip of the clamping arthroscopic resection tool 100 as well as laterally to an extended side 224 so that the first jaw 116 is wider than the neck. The first jaw clamping surface extends distally towards the tip of the clamping arthroscopic resection tool 100, laterally, and then proximally away from the tip of the clamping arthroscopic resection tool 100 in a U or V shape disposed around the slot 208. The shape and size of the first jaw 116 and second jaw 132 are similar to support tissue held between the first jaw 116 and second jaw 132. Also visible are serrations 228 formed on the surface of the first jaw 116 to aid in gripping the tissue.

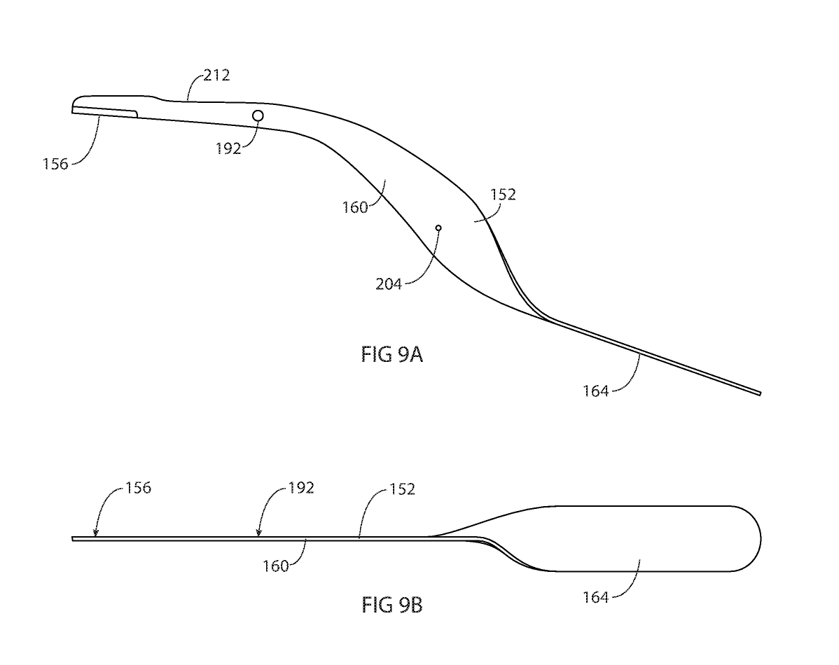

FIGS. 9A and 9B show side and bottom view drawings of the third body member 152. It can be seen how the third body member 152 is reduced in size at a neck portion 212 located between the pivot hole 192 and the cutting blade 156.

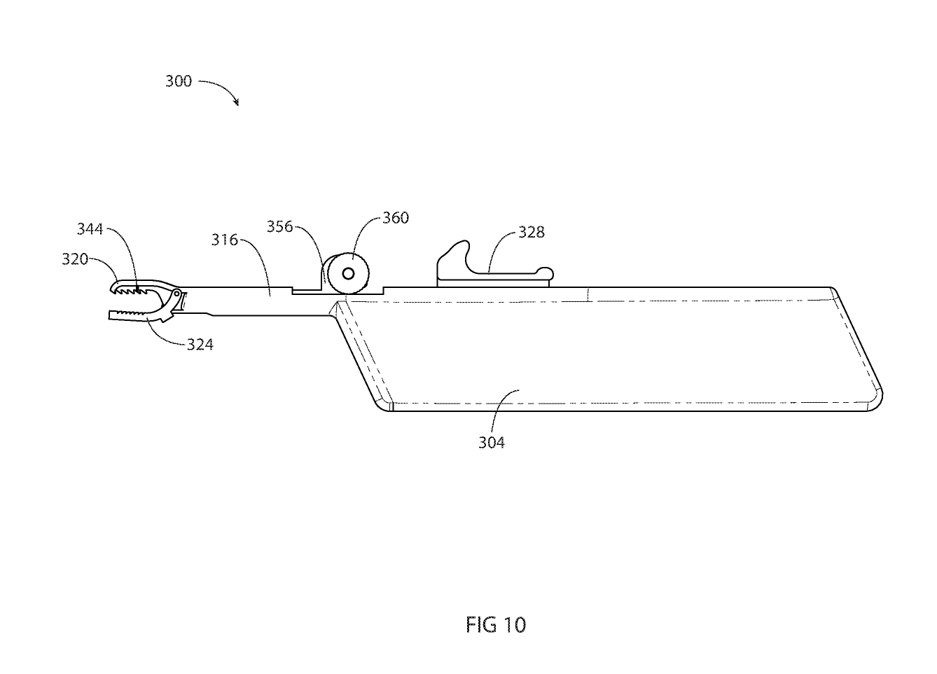

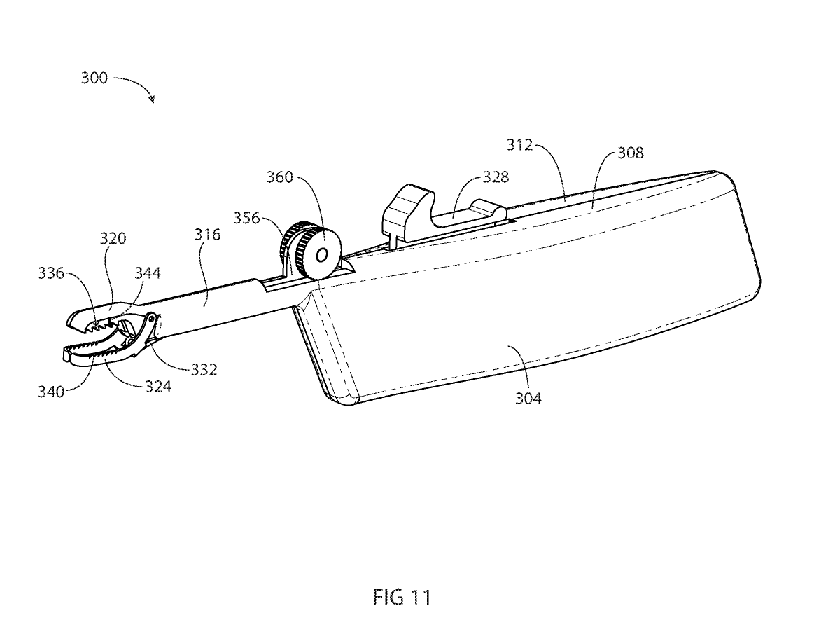

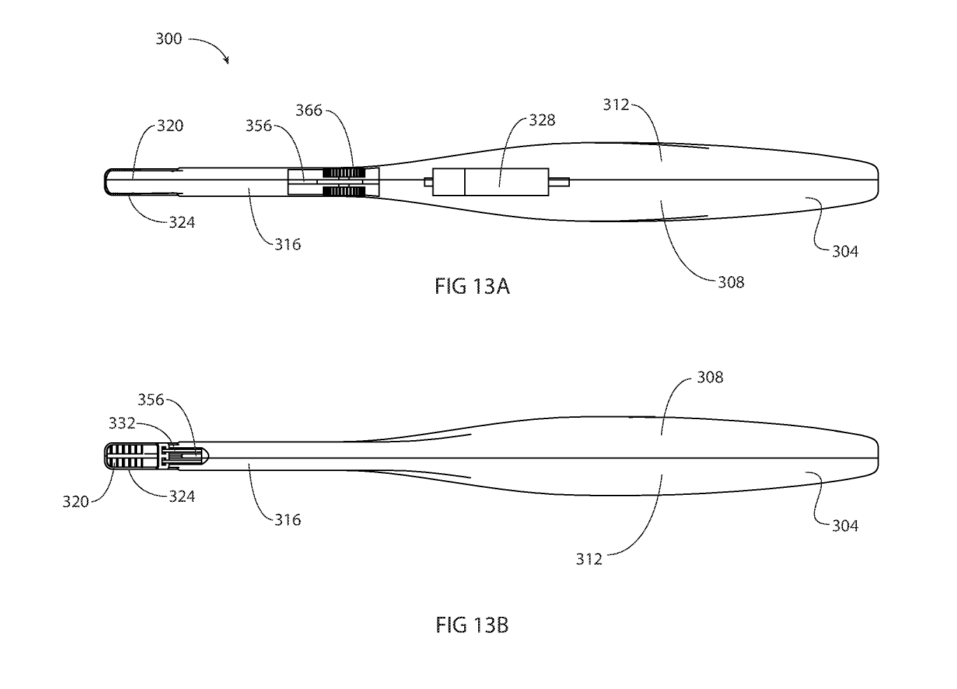

FIGS. 10 through 19C show a disposable clamping arthroscopic resection tool 300. The clamping arthroscopic resection tool 300 is similar to the clamping arthroscopic resection tool 100 discussed above in many aspects of structure and operation and is understood to be used in the manner discussed above and to function in the manner discussed above unless noted otherwise. Both the durable clamping arthroscopic resection tool 100 and the disposable clamping arthroscopic resection tool 300 have a body, a handle portion which allows a surgeon to manipulate and operate the tool, first and second clamping jaws which engage and hold a tissue between the clamping jaws, an indicator mark on the clamping jaws, and a cutting blade which moves between the clamping jaws so that the distal end of the cutting blade is located distally at the indicator mark to cut through the tissue up to the indicator mark.

The clamping arthroscopic resection tool 300 includes a body 304 which may be assembled from left and right halves 308, 312. The body 304 includes an extending neck 316 which forms a distal upper jaw 320. The body, neck, and upper jaw may be integrally formed. A lower jaw 324 is pivotably attached to the neck 316 adjacent the base of the upper jaw 320. The lower jaw 324 is connected to a manual grip such as a button 328 via a pushrod 332. The base of the button 328 is captured by the body 304 and is slidable forwards and backwards. The pushrod 332 is located within the body 304 and neck 316. Sliding the button 328 forwards and backwards moves the lower jaw 324 towards and away from the upper jaw 320 to clamp tissue therebetween. The upper jaw 320 may include serrations 336. Similarly, the lower jaw 324 may include serrations 340. The button 328 may be locked into a position whereby tissue is clamped between the upper jaw 320 and lower jaw 324 via ratcheting teeth on the button 328 and ratcheting teeth on the body 304 which engage each other. An indicator mark 344 is formed on the upper jaw. The indicator mark 344 shows where a cutting blade will pass and shows the extent to which a cutting blade will cut tissue held between the upper jaw 320 and the lower jaw 324.

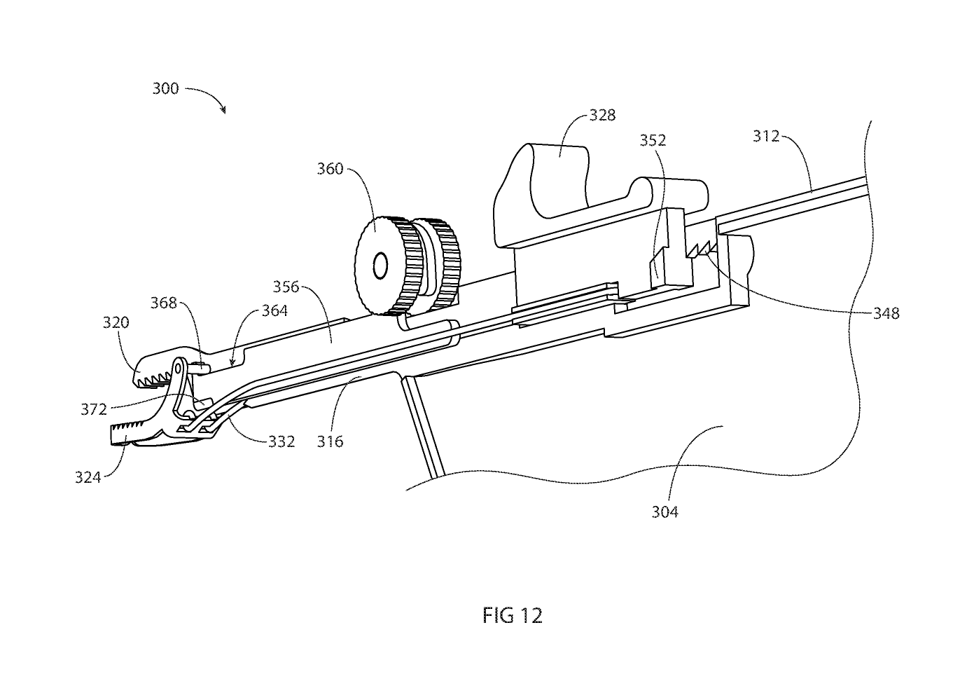

FIG. 12 shows a partial view of a partially disassembled clamping arthroscopic resection tool 300 having a side of the body 304 removed to view internal components. The view focuses on the mechanisms which clamp target tissue and which cut the tissue. It can be seen how the body 304 includes teeth 348 which are formed adjacent a slot in which the button 328 is held. The button has complementary teeth 352. The teeth 348 on the body 304 engage the teeth 352 on the button 328 to prevent proximal motion of the button 328 and thereby hold the button in a distal position in order to clamp tissue between the jaws 320, 324. The button 328 may be pushed downwardly into the body 304 to release engagement between the teeth 348, 352. It can be seen how the pushrod 332 connects to the button 328 and to the lower jaw 324 to transfer the forwards/backwards movement of the button to the lower jaw 324 and thereby create rotary movement of the lower jaw 328 to clamp target tissue between the upper jaw 320 and the lower jaw 324.

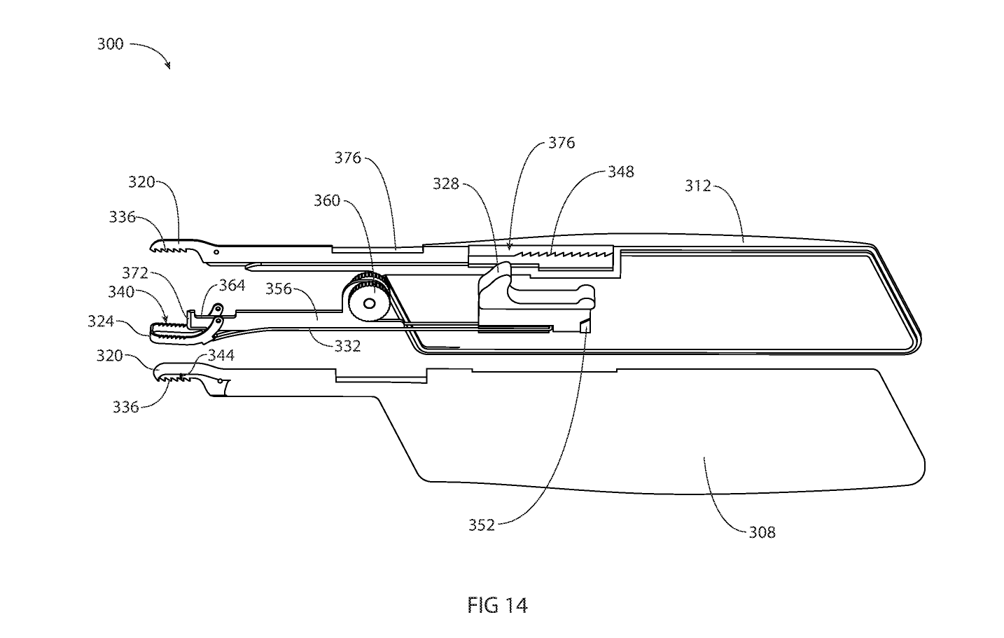

A cutting member 356 is held within the body 304 and extends through the neck 316. The cutting member 356 slides distally and proximally within the neck 316 and body 304. The cutting member includes a finger grip such as thumb wheel 360 to allow a surgeon to move the cutting member distally and proximally by using the finger grip. The cutting member 356 is limited in its travel both distally and proximally, such as by having a notch 364 and associated end walls which engage a pin 368 or other travel limiting device. In the example clamping arthroscopic resection tool 300, the pin 368 is the pivoting pin used to attach the lower jaw 324 to the body 304. Alternatively, the cutting member 356 may include a tab or other structure adjacent the thumb wheel 360 which engages ends of a slot 376 (FIG. 14) in the body 304 to limit the travel of the cutting member.

The cutting member slides between a proximal non-cutting position and a distal cutting position where the distal edge of the forwards facing blade 372 is located at the indicator mark 344. At the proximal position, the cutting member blade 372 is retracted within the neck 316 and does not cut tissue held between the upper jaw 320 and lower jaw 324. In the distal position, the cutting member blade extends between the upper jaw 320 and lower jaw 324 to cut tissue. A slot may be formed down the center of the clamping face of the upper jaw 320 and the center of the clamping face of the lower jaw 324 to allow the sides of the blade 372 to pass therethrough and ensure complete cutting of the target tissue. In a manner similar to that discussed above, the clamping arthroscopic resection tool 300 can be clamped to a desired tissue while using the indicator mark 344 to determine the amount of tissue which will be cut and the amount of tissue which will not be cut, and then cut the tissue with the cutting member 356.

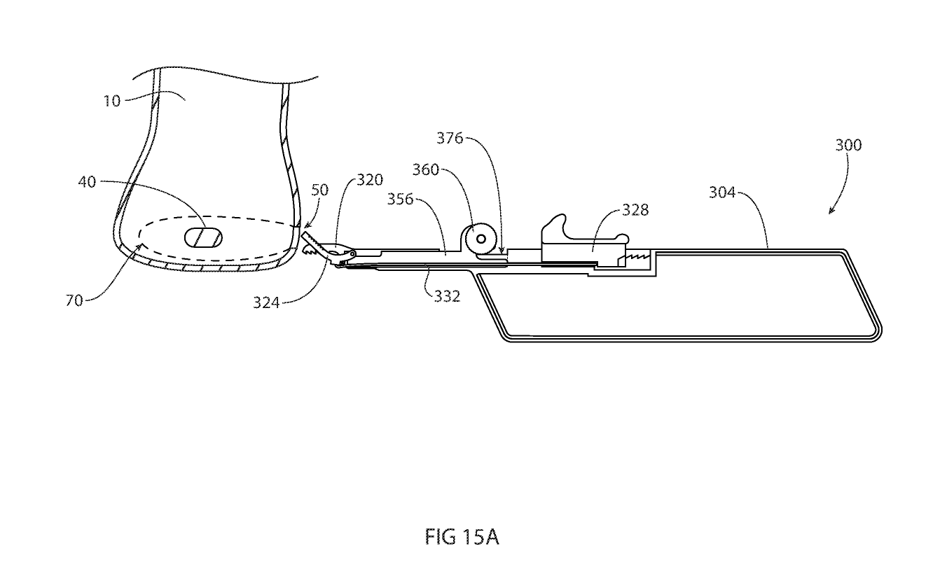

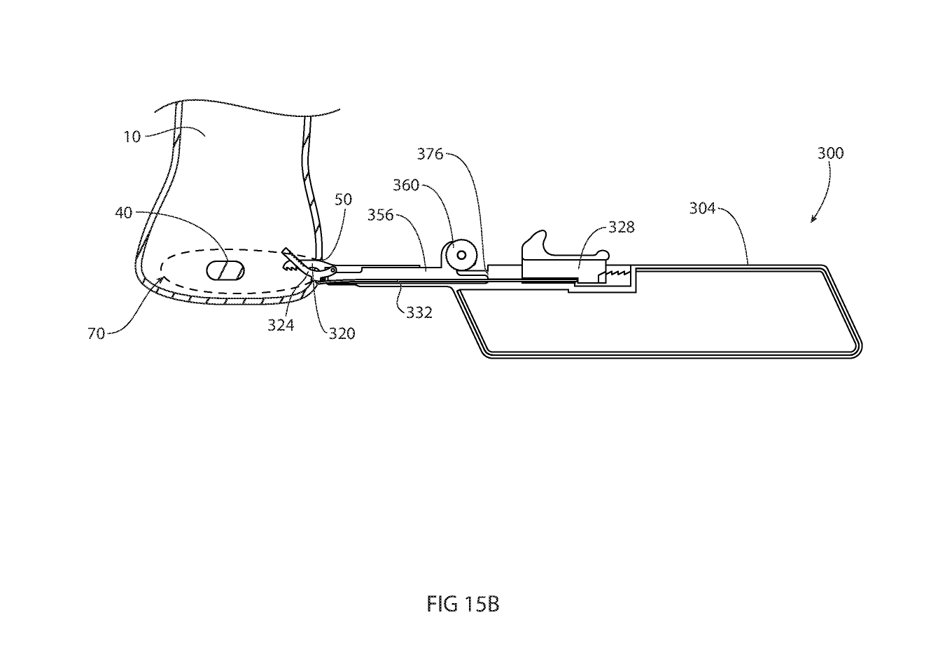

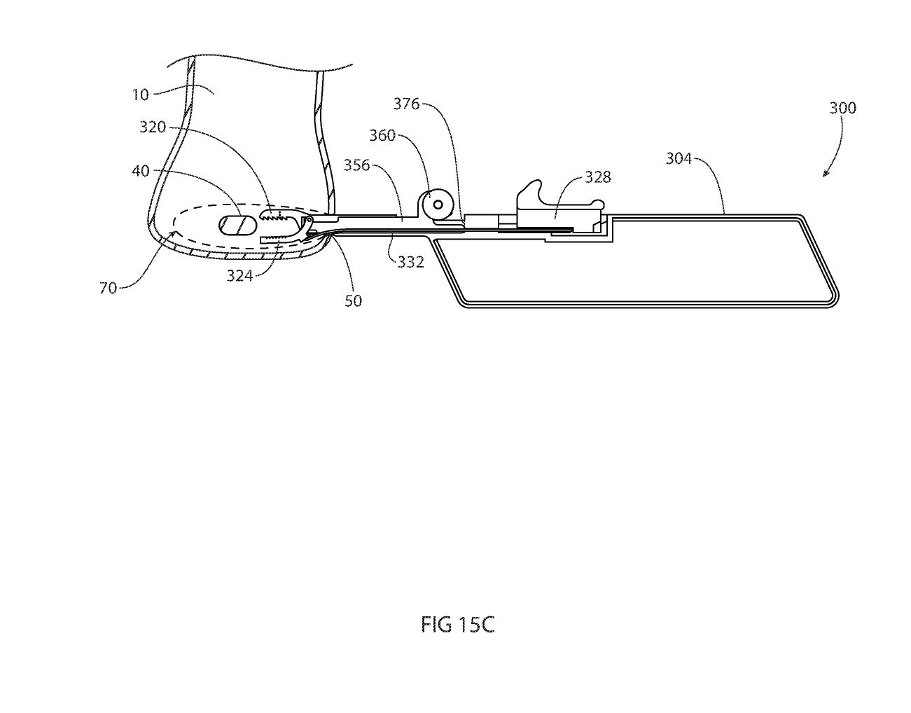

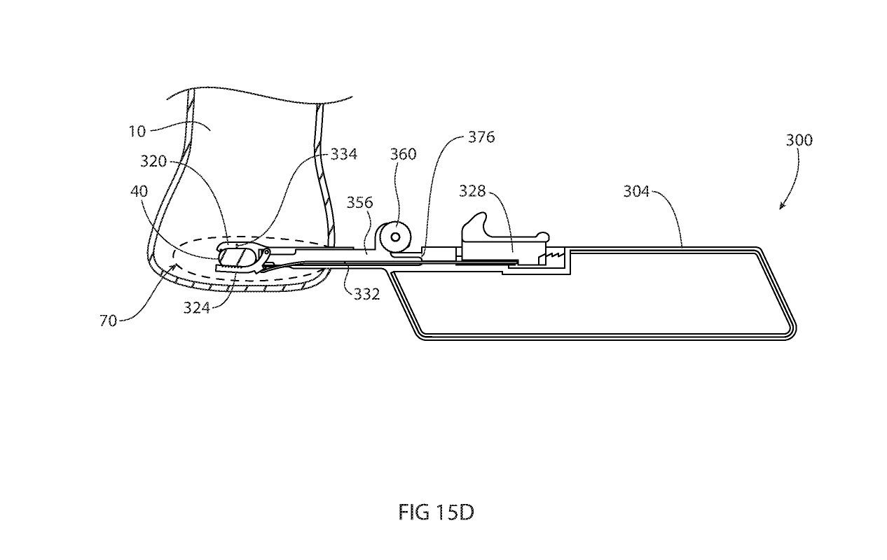

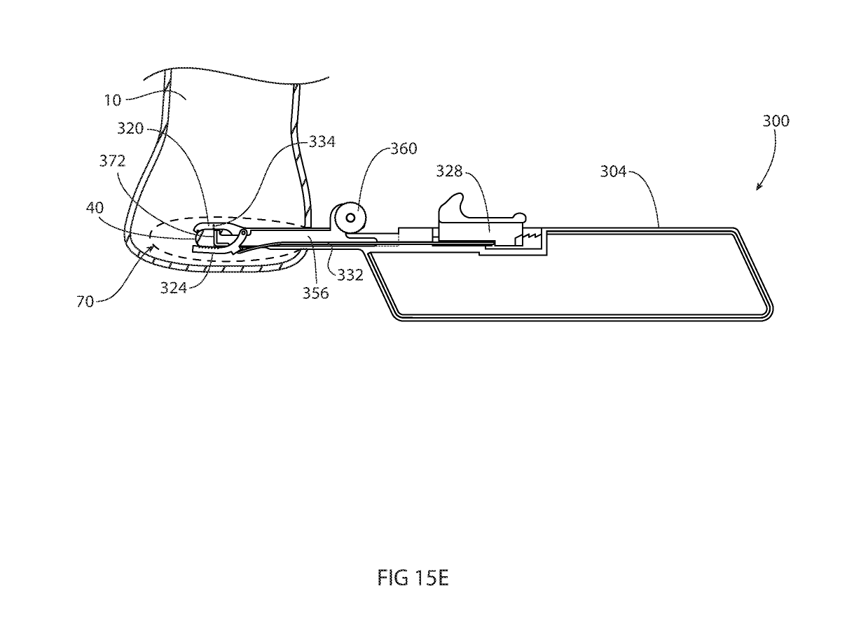

FIGS. 15A through 15E show use of the clamping arthroscopic resection tool 300. The clamping arthroscopic resection tool 300 is used in the manner discussed above unless otherwise noted and some details of its use have not been duplicated here. As shown in FIG. 15A, the clamping arthroscopic resection tool 300 may be brought into a position adjacent the first incision 50 with its jaws 320, 324 closed. The clamping arthroscopic resection tool 300 may be inserted into the operative cavity 70 through the first incision 50 as shown in FIG. 15B. The clamping arthroscopic resection tool 300 may be brought to a position adjacent the plantar fascia 40 and the jaws 320, 324 of the clamping arthroscopic resection tool 300 may be opened to receive the plantar fascia 40. As shown in FIG. 15D, the indicator mark 344 may be seen through the arthroscope 80 and used to assist the surgeon in placing the jaws 320, 324 around the plantar fascia 40 and dividing the plantar fascia 40 into a cut portion and uncut portion. The surgeon may then move the button 328 to clamp the plantar fascia 40 between the upper jaw 320 and the lower jaw 324. As shown in FIG. 15E, the surgeon may then use the finger grip 360 to move the blade 372 through the plantar fascia 40 and cut the plantar fascia 40 up to the indicator mark 344. The surgeon may then retract the cutting blade 372 and release the clamping jaws 320, 324. After completing any other necessary cuts, the surgeon may remove the clamping arthroscopic resection tool 300 from the first incision 50.

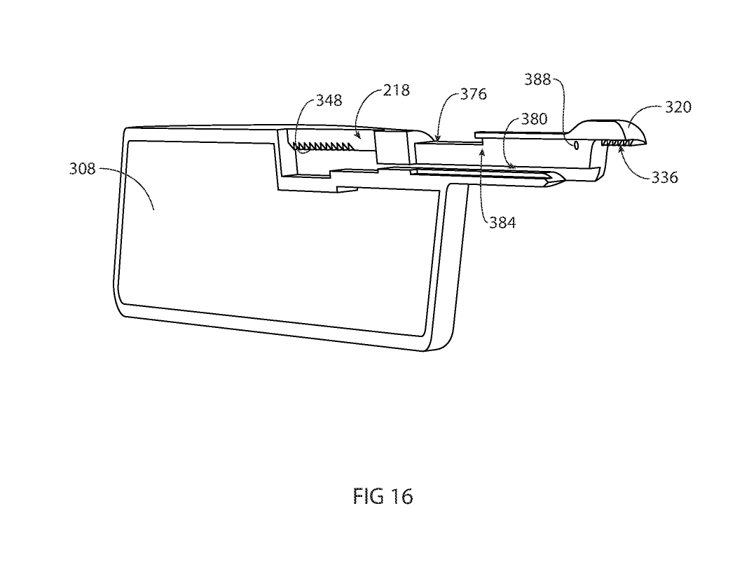

FIG. 16 shows a perspective view of the left half 308 of the body 304. The body half 308 includes a slot 380 to house the pushrod 332, a channel or slot 384 to house the cutting member 356, a slot to accept the button 328 and permit its movement, teeth 348 to limit the movement of the button 328, and a hole 388 to pivotably mount the lower jaw 324.

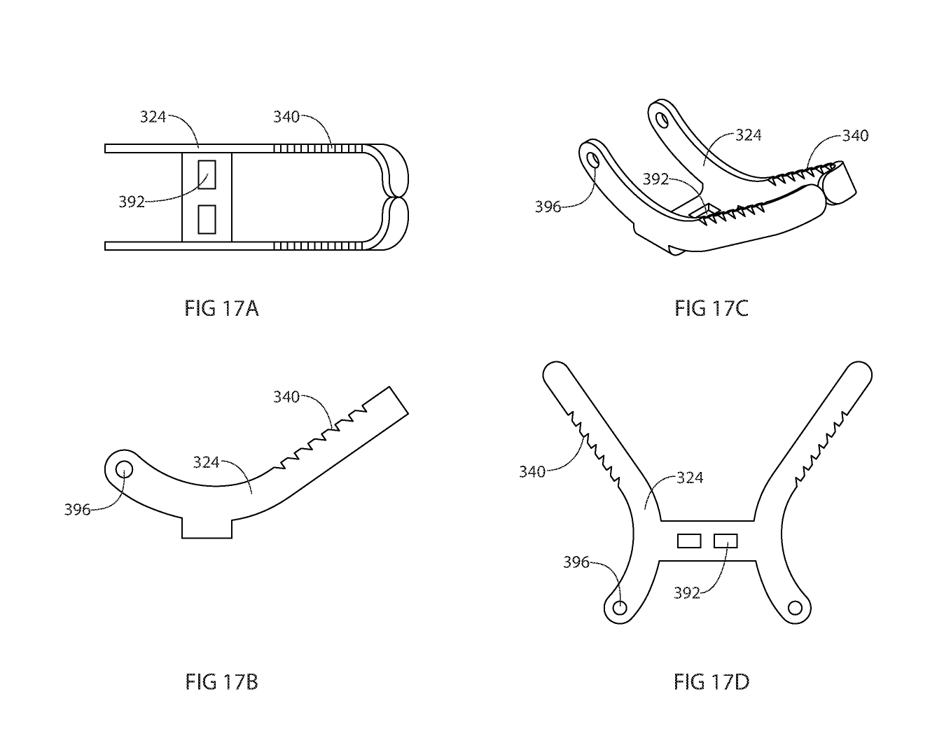

FIGS. 17A through 17D show top, side, perspective, and sheet metal cutout views of the lower jaw 324. The lower jaw 324 includes a hole or holes 392 to receive the pushrod 332 and holes 396 to accept the pin 368 used to mount the lower jaw 324 to the body 304.

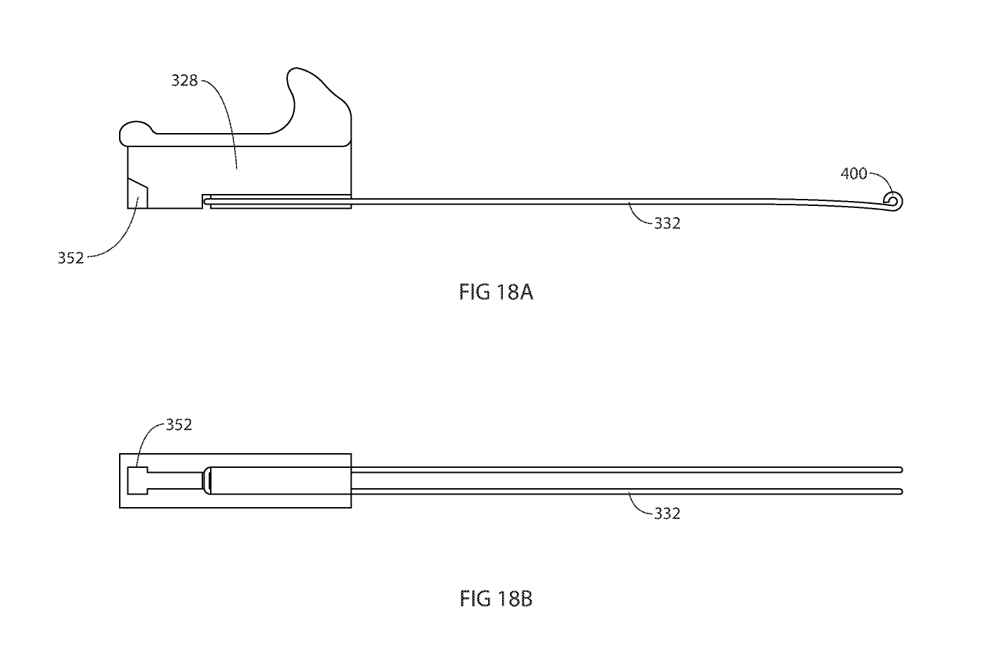

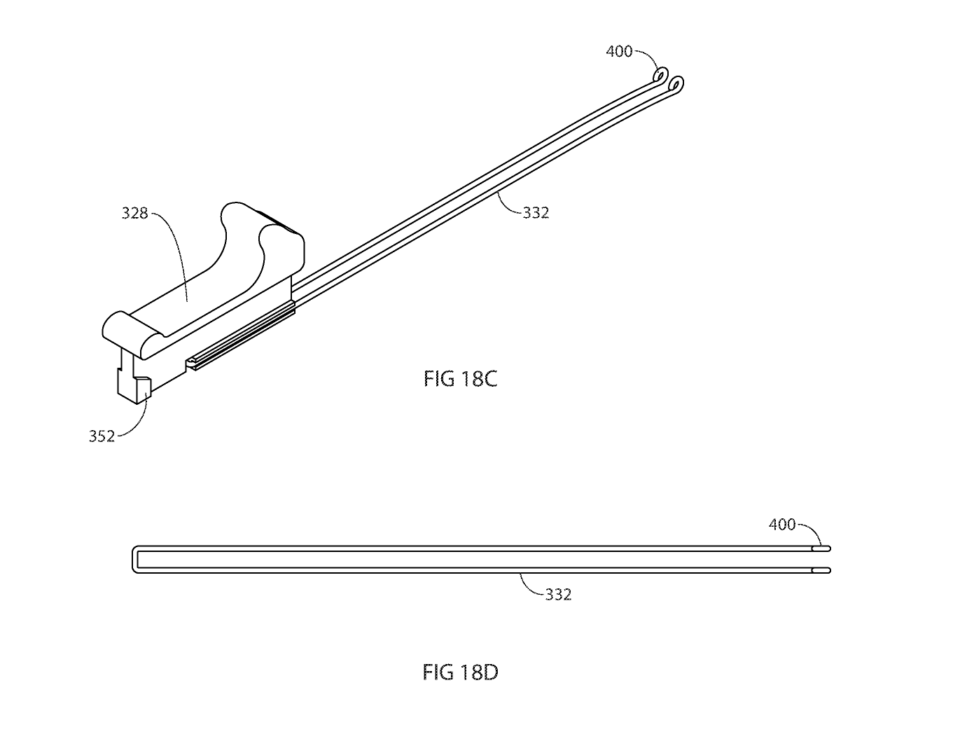

FIGS. 18A through 18D show side, bottom, and perspective views of the clamp actuation button 328 and associated pushrod 332 and a top view of the pushrod 332. The button 328 is contoured to permit easy manual manipulation with the thumb. The pushrod 332 is attached to the button 328, but these may be formed as a single piece. The distal end(s) of the pushrod 332 is formed with a hole 400 or other attachment structure to engage the lower jaw 324.

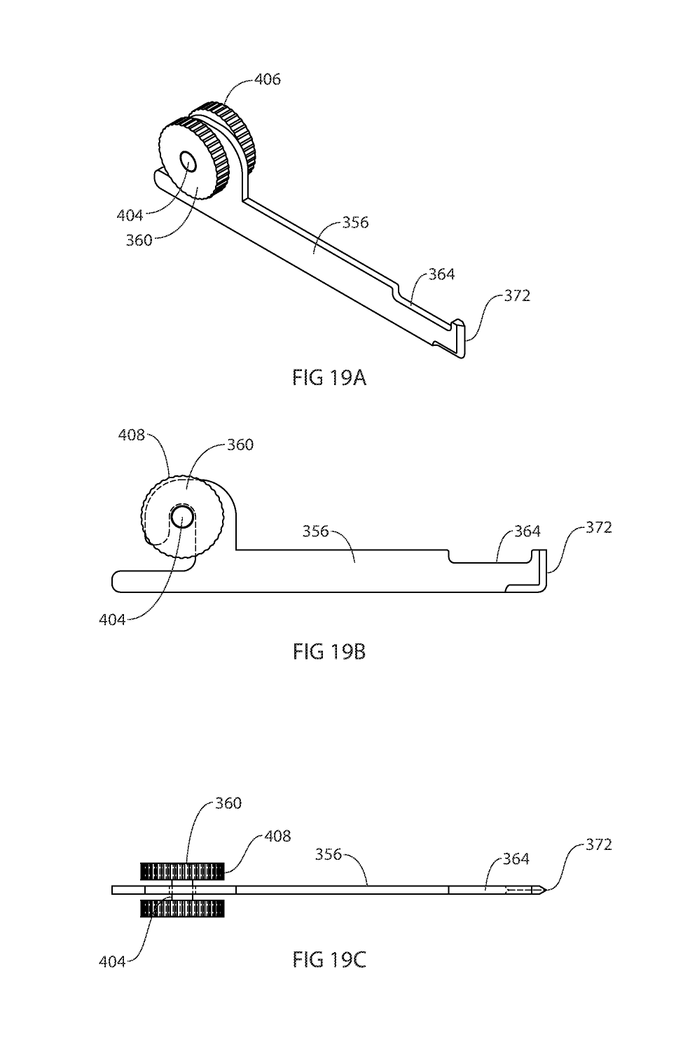

FIGS. 19A through 19C show perspective, side, and top views of the cutting member 356. The cutting member 356 includes a finger grip such as a thumbwheel 360 with grip texture 408 which is rotatably attached to the cutting member body with a pin 404. The thumbwheel may be rolled against the body of the clamping arthroscopic resection tool 300 with the thumb to move the cutting member 356 forwards and backwards to cut tissue and retract the blade 372.

In the manner discussed above, the clamping arthroscopic resection tool may be used to perform other tissue releases and surgical cuts. It is advantageous in these situations as it allows for more accurate cutting of the tissue. The clamping arthroscopic resection tool is advantageous as it allows a surgeon to release the plantar fascia and other soft tissues in a manner which allows for easier observation of the dimensions of the tissue, improved visual separation of the tissue into a desired cut portion and a non-cut portion, and cutting of the tissue precisely to an indicator mark placed at a previously determined location along the tissue.

The above description of illustrated examples of the present invention, including what is described in the Abstract, are not intended to be exhaustive or to be limitation to the precise forms disclosed. While specific examples of the invention are described herein for illustrative purposes, various equivalent modifications are possible without departing from the broader scope of the present claims. Indeed, it is appreciated that specific example dimensions, materials, etc., are provided for explanation purposes and that other values may also be employed in other examples in accordance with the teachings of the present invention.

What is claimed is:

1. A clamping arthroscopic resection tool comprising:
a first body member comprising:
a first clamping jaw disposed at a distal end of the first body member;
a shank attached to the first clamping jaw and extending proximally from the first clamping jaw; and
a handle attached to a proximal end of the shank;
a second body member comprising:
a second clamping jaw disposed at a distal end of the second body member;
a shank attached to the second clamping jaw and extending proximally from the second clamping jaw; and
a handle attached to a proximal end of the shank;
a third body member comprising:
a cutting blade disposed at a distal end of the third body member;
a shank attached to the cutting blade and extending proximally from the cutting blade; and
a finger grip attached to a proximal end of the shank;
wherein the first body member, the second body member, and the third body member are pivotably attached to each other at a pivot;
wherein the cutting blade is movable independent of the first clamping jaw and the second clamping jaw;
wherein the clamping arthroscopic resection tool is attached to a target body tissue by holding the target body tissue between the first clamping jaw and the second clamping jaw; and
wherein the cutting blade is movable through the target body tissue to cut a portion of the target body tissue held between the first clamping jaw and the second clamping jaw.

2. The tool of claim 1, wherein a width of the target body tissue is held between the first clamping jaw and the second clamping jaw, and wherein the cut portion of the target body tissue is less than the width of the target body tissue.

3. The tool of claim 1, further comprising an indicator mark formed on a side of the second clamping jaw, wherein a distal end of the cutting blade is disposed adjacent the indicator mark, and wherein the cutting blade moves through the target body tissue to cut the target body tissue located proximally from the indicator mark and to not cut target body tissue located distally from indicator mark.

4. The tool of claim 1, wherein the first body member further comprises a neck located between the first clamping jaw and the shank and disposed distally of the pivot, and wherein the second body member further comprises a neck located between the second clamping jaw and the shank and disposed distally of the pivot.

5. The tool of claim 1, wherein the second clamping jaw comprises a slot formed therethrough and wherein the cutting blade passed through the slot to pass between the first clamping jaw and the second clamping jaw and cut the target tissue.

6. The tool of claim 5, wherein the first clamping jaw comprises a slot formed at least partially into a clamping face, and wherein the cutting blade passes into the slot after passing through target tissue to cut the target tissue.

7. The tool of claim 1, wherein the third body member moves between a first position wherein the cutting blade does not contact target body tissue held between the first clamping jaw and the second clamping jaw and a second position wherein the cutting blade is located beyond a clamping face of the second clamping jaw towards the first clamping jaw to thereby cut a target body tissue located between the first clamping jaw and the second clamping jaw.

8. The tool of claim 7, further comprising a biasing element attached to the second body member and the third body member, and wherein the biasing element biases the third body member into the first position.

9. The tool of claim 1, further comprising a first clamp member attached to the first body member and a second clamp member attached to the second body member, and wherein the first clamp member engages the second clamp member to prevent movement of the first clamping jaw away from the second clamping jaw to thereby hold a target body tissue between the first clamping jaw and the second clamping jaw.

10. The tool of claim 1, wherein the cutting blade is movable independent of both the first clamping jaw and the second clamping jaw.

11. A clamping arthroscopic resection tool comprising:
a body comprising:
a handle formed on a proximal end of the body;
a neck extending distally from the handle; and
a first clamping jaw attached to the neck;
a second clamping jaw pivotably attached to the body such that the second clamping jaw may selectively pivot towards or away from the first clamping jaw;
a manual grip attached to the body and operatively connected to the second clamping jaw;
wherein the manual grip is movable between a first position and a second position to thereby move the second clamping jaw towards the first clamping jaw to thereby clamp a target body tissue between the first clamping jaw and the second clamping jaw;
a cutting blade attached to the body and movable relative thereto;
a finger grip operatively connected to the cutting blade;
a stop to prevent movement of the cutting blade;
wherein the finger grip is movable to move the cutting blade to a position between the first clamping jaw and the second clamping jaw to cut a target body tissue held between the first clamping jaw and the second clamping jaw; and
wherein the cutting blade contacts the stop to prevent distal movement of the blade between the first clamping jaw and the second clamping jaw.

12. The tool of claim 11, wherein the cutting blade moves distally from a position adjacent the pivot to a position between the first clamping jaw and the second clamping jaw to cut a target body tissue held between the first clamping jaw and the second clamping jaw.

13. The tool of claim 11, further comprising an indicator mark formed on a side of at least one of the group consisting of the first clamping jaw and the second clamping jaw; and wherein the cutting blade moves from a first position where the cutting blade is located proximally of the first clamping jaw and the second clamping jaw and does not cut a target body tissue held between the first clamping jaw and the second clamping jaw, and a second position where the cutting blade is located distally from the first position and is located between the first clamping jaw and the second clamping jaw to cut a target body tissue held between the first clamping jaw and the second clamping jaw, and wherein a distal cutting edge of the cutting blade is located adjacent the indicator mark when the cutting blade is in the second position.

14. The tool of claim 11, wherein the cutting blade is movable independent of both the first clamping jaw and the second clamping jaw.

15. A method of performing a soft tissue release in a body comprising:
creating an incision through skin of the body;
selecting a clamping resection tool comprising:
a body;
a handle attached to the body;
a first clamping jaw attached to a distal end of the body;
a second clamping jaw pivotably attached to the body such that the second clamping jaw is pivotable movable towards the first clamping jaw and the second clamping jaw;
a manual grip operatively connected to the second clamping jaw and movable to thereby move the first clamping jaw towards the second clamping jaw;
an indicator mark located on an indicator surface of at least one of the group consisting of the first clamping jaw and the second clamping jaw, wherein the indicator surface is not a clamping surface;
a cutting blade connected to the body and movable independent of the first clamping jaw and the second clamping jaw between a first, non-cutting position and a second, cutting position, wherein a distal cutting edge of the cutting blade is aligned with the indicator mark when the cutting blade is disposed in the second, cutting position; and
a finger grip operatively connected to the cutting blade and movable to thereby move the cutting blade between the first, non-cutting position and the second, cutting position;
moving the distal end of the clamping resection tool through the incision;
positioning a target body tissue between the first clamping jaw and the second clamping jaw;
adjusting the position of the target body tissue relative to the first clamping jaw and the second clamping jaw by using the indicator mark to visually separate the target body tissue into a first portion which is to be cut and a second portion which is not to be cut;
moving the manual grip to thereby clamp the target body tissue between the first clamping jaw and the second clamping jaw; and
moving the finger grip to thereby move the cutting blade from the first, non-cutting position to the second, cutting position and thereby cut the target body tissue which is located between the first clamping jaw and the second clamping jaw proximally of the indicator mark and to not cut the target body tissue which is located between the first clamping jaw and the second clamping jaw distally of the indicator mark.

16. The method of claim 15, wherein the method comprises selecting a clamping resection tool which comprises:
a second body member;
a handle attached to the second body member;
wherein the second clamping jaw attached to a distal end of the second body member;
wherein the body is pivotably attached to the second body member; and
wherein the cutting blade is pivotably attached to the body and the second body member.

17. The method of claim 15, wherein the method further comprises locking the second clamping jaw relative to the first clamping jaw with a locking member to prevent movement of the clamped target body tissue relative to the first clamping jaw and the second clamping jaw.

18. The method of claim 15, wherein the method further comprises:
creating a second incision through skin of the body adjacent the first incision;
inserting an arthroscope through the second incision; and
using the arthroscope to visualize operation of the clamping resection tool.

19. The method of claim 15, wherein the step of positioning a target body tissue between the first clamping jaw and the second clamping jaw more specifically comprises positioning a plantar fascia tissue between the first clamping jaw and the second clamping jaw.
